(19) United States
(12) Patent Application Publication
(10) Pub. No.: US 2008/0220094 A1
(43) Pub. Date: Sep. 11, 2008

(54) Compositions and methods for the treatment and/or prevention of osteoporosis

(75) Inventors: Gregory Eugene Bobyock, Upper Montclair, NJ (US); Judith Diane Kendler Moreines, Edgewater, NJ (US); Lynda Sun Lee, Murray Hill, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/978,794

(22) Filed: Oct. 30, 2007

Related U.S. Application Data
(60) Provisional application No. 60/855,942, filed on Nov. 1, 2006.

(51) Int. Cl. A61K 33/06; A61K 31/122; A61K 31/592

(52) U.S. Cl. 424/682; 514/681; 514/168

(57) ABSTRACT

The invention of this application relates to compositions and methods for the treatment and/or prevention of osteoporosis; improving and/or enhancing bone formation; and prevention and/or reducing bone mineral density loss. More particularly, the invention relates to a composition comprising vitamin K, in any form and betacryptoxanthin. A further aspect of this invention is a composition comprising vitamin K, in any form, betacryptoxanthin, and/or vitamin D and/or calcium.

This application claims priority from copending U.S. provisional application No. 60/855,942, filed Nov. 1, 2006, the entire disclosure of which is hereby incorporated by reference.

### FIELD OF THE INVENTION

The invention of this application relates to compositions for the treatment and/or prevention of osteoporosis. More particularly, one aspect of the invention relates to a composition comprising vitamin K², and betacryptoxanthin. The compositions may optionally contain vitamin D, in any form and or calcium. These compositions are useful for the treatment and/or prevention of osteoporosis and increased bone health. The invention further relates to methods of treating and/or preventing osteoporosis with the compositions disclosed herein.

### BACKGROUND

Throughout life, old bone is continuously removed by bone-resorbing osteoclasts and replaced with new bone, which is formed by osteoblasts. This cycle is called the bone-remodelling cycle and is normally highly regulated, i.e. the functioning of osteoclasts and osteoblasts is linked such that in a steady state the same amount of bone is formed as is resorbed.

The bone-remodelling cycle occurs at particular areas on the surfaces of bones. Osteoclasts which are formed from appropriate precursor cells within bones resorb portions of bone; new bone is then generated by osteoblastic activity. Osteoblasts synthesize the collagenous precursors of bone matrix and also regulate its mineralization. The dynamic activity of osteoblasts in the bone remodeling cycle to meet the requirements of skeletal growth and matrix and also regulate its maintenance and mechanical function is thought to be influenced by various factors, such as hormones, growth factors, physical activity and other stimuli. Osteoblasts are thought to have receptors for parathyroid hormone and estrogen. Osteoclasts adhere to the surface of bone undergoing resorption and are thought to be activated by some form of signal from osteoblasts.

It is considered that various bone diseases occur because, for example, calcium content of bones is decreased by the bone metabolism and insufficient osteogenesis. Typical bone diseases are, for example, fracture, osteomalacia, osteopenia, osteoporosis, back pain and low back pain. Osteoporosis has a pathology caused by bone mass decrease as the balance of the bone resorption and the bone formation is lost by aging and, accordingly, the bone resorption is relatively increased to reduce the bone mass. As a result, bone strength is decreased by the change in the fine structure of the bones thereby causing fracture. Particularly in females, the bone mass is rapidly decreased after menopause, oophorectomy, etc. Osteoporosis is characterized generally by a loss of bone density. Thinning and weakening of the bones leads to increased fracturing from minimal trauma. The most prevalent fracturing in post-menopausal osteoporotics is of the wrist and spine. Senile osteoporosis, is characterized by a higher than average fracturing of the femur.

Furthermore, in osteoporosis, there is a relative increase in osteoclastic activity which may cause a reduction in bone density and mass. Osteoporosis is the most common of the metabolic bone diseases and may be either a primary disease or may be secondary to another disease or other diseases. Whilst osteoporosis as a therapeutic target has been of, and continues to, attract a great deal of interest, tight coupling between the osteoblastic and osteoclastic activities of the bone remodeling cycle make the replacement of bone already lost an extremely difficult challenge. Consequently, research into treatments for prevention or prophylaxis of osteoporosis (as opposed to replacement of already-lost bone) has yielded greater results to date.

Presently, there exists the need for medicines to effectively treat and/or prevent the occurrence of osteoporosis. However, many of the already approved treatments have significant side effect profiles. Therefore, there exists a need in the art for medicines to effectively treat and/or prevent the occurrence of osteoporosis without the unwanted side effects. These side effects are a leading cause for the lack of compliance with current treatment regimes. Such current treatment regimes include bisphosphonates, estrogen therapies, calcitonin.

Vitamin K is an essential vitamin and the family name for a group of highly lipophilic compounds that have a common chemical ring structure (naphthoquinone), but differ with respect to the side chain at the 3-position. There are two main categories of vitamin K, i.e., vitamin K1 (phylloquinone) and vitamin K2. Vitamin K1 is the major form of vitamin K in the diet and is synthesized by plants; it is found in green leafy vegetables and certain plant oils; e.g., canola and soybean. Bacteria synthesize vitamin K2 as a series of menaquinones. Vitamin K2 is also found in animal organs and muscle as well as in fermented products, e.g., cheese, curd cheese, and natto.

Vitamin K was first reported has having an effect upon bone fracture healing in 1960 and since that time further studies have suggested that vitamin K2 both increases bone formation and decreases bone resorption. Studies have also demonstrated that postmenopausal bone loss and osteoporosis is associated with vitamin K deficiency.

Beta Cryptoxanthin, (hereinafter referred to as “BCX”), is a carotenoid pigment found in high concentration in citrus fruits and juices. It has been shown to directly stimulate bone formation and directly inhibit bone resorption by stimulating synthesis of osteoblasts and inhibiting synthesis of osteoclasts, respectively. These effects are mediated via genetic transcription and protein synthesis.

Adequate calcium ingestion and absorption is crucial for the maintenance of bone mass. The average person loses 400 to 500 mg of valcium per day. If an individual's diet is low in calcium, there may not be sufficient amounts of calcium available in the blood to be returned to the bones to maintain strong bones and total body health. Adequate calcium intake throughout life, particularly for women during their first three decades when peak bone mass is achieved, is a significant factor in determining if osteoporosis will develop later in life. Additionally, adult women benefit from calcium supplementation even if peak bone mass has been attained at an earlier age. Research demonstrates that calcium supplementation by itself has not been successful in preventing or curing the disease. Furthermore, calcium sources vary in bioavailability and effectiveness in preventing bone loss.

### SUMMARY OF THE INVENTION

The invention of this application relates to compositions and methods for the treatment and/or prevention of osteoporosis; improving and/or enhancing bone formation; and prevention and/or reducing bone mineral density loss. More particularly, the invention relates to a composition comprising vitamin K2 and BCX. In a preferred embodiment, the composition comprises MK4 or MK7. In an even more preferred embodiment, the composition comprises MK7. A further aspect of this invention is a composition comprising vitamin K2 betacryptoxanthin, and/or vitamin D and/or calcium.

### DESCRIPTION OF THE DRAWINGS

FIG. 1 is the chemical structure of Vitamin K and Vitamin K2

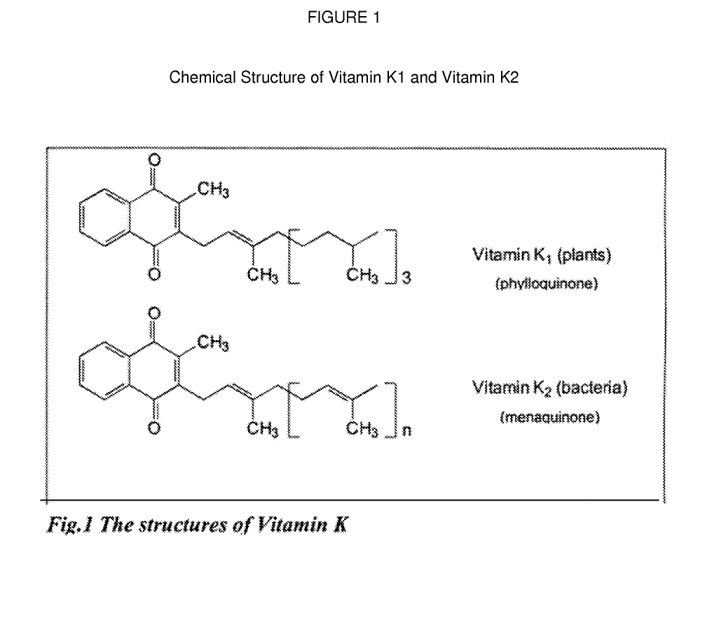

### DETAILED DESCRIPTION OF THE INVENTION

Vitamin K is the family name for a group of highly lipophilic compounds that have a common chemical ring structure (naphthoquinone), but differ with respect to the side chain at the 3-position (i.e., one or more isoprenoid side chains attached). There are two main categories of vitamin K, i.e., vitamin K1 (phylloquinone) and vitamin K2 (menaquinone). The menaquinone homologs are characterized by the number of isoprene residues comprising the side chain. Menaquinones, (also abbreviated as MK-n, whereby n represents the number of isoprenoid side chains), with up to 15 isoprene residues have been characterized. Thus, menaquinone-4, abbreviated MK-4, possesses four isoprene residues in the side chain. Menaquinone-7, abbreviated MK-7, possesses seven isoprene units in the side chain. The menaquinones may also be designated by the number of carbons in the side chain. An isoprene residue contains five carbons. Thus, menaquinone-4 is also called vitamin K2 and menaquinone-7 is also called vitamin K2. Menaquinone-4 is also known as menatetrenone.

Vitamin K1 is the major form of vitamin K in the diet and is synthesized by plants; it is found in green leafy vegetables and certain plant oils; e.g., canola and soybean. Bacteria synthesize vitamin K2 as a series of menaquinones. Vitamin K2, also known as menatetrenone, is found in animal organs and muscle as well as in fermented products, e.g., cheese, curd cheese, and natto.

Physiological data support vitamin K2 preferential activity over K1 in bone tissues. Vitamin K1 has been shown to be preferentially transported and taken up by the liver; whereas, vitamin K2 is preferentially transported and concentrated in bone tissues. The greater efficacy of MK-7 compared with MK-4 derives from the greater half-life of MK-7. Therefore, in a preferred embodiment, the composition comprises vitamin K2; in an even more preferred embodiment, the composition comprises vitamin K2, MK-7.

Vitamin K2 is superior to vitamin K1 for promoting bone health. Both vitamins K1 and K2 are capable of promoting carboxylation of osteocalcin thereby allowing the osteocalcin to tightly bind hydroxyapatite in bone, thereby promoting mineralization. However, vitamin K2, but not K1, also promotes inhibition of bone resorption. This effect is based on the isoprenyl residues in the aliphatic side chain. In this regard, it is believed that MK-7 is more effective than MK-4.

Furthermore, vitamin K2 is more effective than vitamin K1 in increasing bone mineral density (“BMD”) and thereby reducing fracture risk. It is believe that, at equivalent doses, vitamin K2 is more effective than vitamin K1 at reducing under-carboxylated osteocalcin, which is directly associated with reduced BMD and increased fracture risk. Additionally, vitamin K2 MK-7 is more effective than vitamin K2 MK-4 at equal doses for reducing under carboxylated osteocalcin. The compositions of the invention comprise a therapeutically effective amount of vitamin K2 in either the MK4 or MK7 form. In a preferred embodiment, the composition comprises a therapeutically effective amount of vitamin K2 MK7.

Methods of making vitamin K are well known in the art. Furthermore, the methods are also known for the preparation of structurally related or similar compounds, for example vitamin K2. Some of these methods have been reviewed in EP-A-0243849.

BCX has been shown to have a stimulatory effect on proliferation, differentiation and mineralization due to enhancement of the gene expression of proteins. BCX has been reported to have osteogenesis promoting effect and thus preventing and/or treating bone diseases. BCX accelerates the synthesis of protein in the bone and thus effectively increases the bone mass. Moreover, BCX has been reported to have a potent inhibitory effect on osteoclast like cell formation, and thus inhibit osteoclastic bone formation. Accordingly, BCX accelerates the osteogenesis and also inhibits the bone resorption to exhibit the effect of keeping and/or increasing BMD. Methods of making and/or isolating BCX are well known in the art, see for example, U.S. Pat. No. 6,911,564.

Without being bound to any theory, the mechanism of BCX is complimentary to vitamin K², preferably vitamin K2 MK4 or MK7. Hence a composition comprising vitamin K² MK4 or MK7 and BCX provides for synergistic effects thereby improving bone health and the biomarkers of bone health e.g., prevention of the loss of bone mineral density, reduction of bone fracture, increase in bone strength, reduced undercarboxylated osteocalcin, reduced resorption and increased bone formation. Such a combination can be used for the prevention and/or treatment of bone loss; increasing bone density; increasing bone strength; preventing and/or reducing bone fractures; increasing biomarkers of bone formation, including but not limited to increasing bone-specific alkaline phosphatase, osteocalcin, reducing undercarboxylated osteocalcin; and reducing the biomarkers of bone resorption.

The compositions described herein are for use in human consumption. One skilled in the art would appreciate that the therapeutically effective amounts of the composition hereinbefore described depends on the usual factors such as the nature and severity of the disorder being treated, the weight of the patient, the specific compound(s) of choice, and considerations and preferences of the prescriber. In one embodiment, the amount of vitamin K2 to be administered usually will be in the range of micrograms up to about 100 mg or more per dose. In a more preferred embodiment, the amount of vitamin K2 present in the composition is in the range of about 10 μg to about 150 μg. In an even more preferred embodiment, the amount of vitamin K2 present in the composition is in the range of about 40 μg to about 60 μg. In the most preferred embodiment, the amount of vitamin K2 present is about 50 μg. The composition of the invention also comprises BCX. In a preferred embodiment, the composition of the invention comprises from about 10 μg to about 6 mg of BCX; in a more preferred embodiment, the composition comprises from about 0.5 mg to about 3 mg of BCX. In yet an even more preferred embodiment composition comprises 2.5 mg of BCX

In yet another embodiment, the composition comprising vitamin K2 and BCX act synergistically such that the amount of vitamin K2 and/or BCX is reduced up to about 90%.

The composition may also contain calcium. Calcium is never found in free form; accordingly one skilled in the art would appreciate that any calcium salt can be used in the compositions of the invention.

As mentioned earlier, vitamin K is capable of promoting carboxylation of osteocalcin thereby allowing the osteocalcin to tightly bind hydroxyapatite in bone, thereby promoting mineralization. Calcium exists in bone primarily in the form of hydroxyapatite (Ca₁₀ (PO₄)₆ (OH)₂). Hydroxyapatite comprises approximately 40% of the weight of bone. Accordingly, in one embodiment, the composition of the invention comprises calcium. In a preferred embodiment, the composition comprises up to about 2000 mgs of calcium. In an even more preferred embodiment, the composition comprises calcium in the range of about 600 mg to about 1200 mg of calcium.

In yet another embodiment, the composition comprising vitamin K2, BCX and calcium act synergistically such that the amount of vitamin K2, BCX, and/or calcium is reduced up to about 90%.

The composition may also contain vitamin D. Vitamin D includes, but is not limited to include vitamin D, cholecalciferol (D₃), ergocalciferol (D₂) and its biologically active metabolites and precursors such as, 1.alpha 25-(OH)₂ vitamin D; 25 OH vitamin D, its biological precursor; and 1.alpha. hydroxy vitamin D, and analogues of the dihydroxy compound. These materials promote intestinal absorption of calcium, contribute to plasma calcium regulation by acting on the remodeling processes of accretion and resorption and stimulate reabsorption of calcium by the kidney.

Vitamin D synthesizes transporter proteins that take up calcium from the GI tract. It is also involved in the synthesis of osteocalcin from the osteoblasts. BCX stimulates the synthesis of osteoblasts. In the presence of osteoblasts, vitamin D then synthesizes osteocalcin. The osteocalcin is carboxylated by vitamin K, in any form, to bind valcium and to deposit it into the hydroxyapatite.

Furthermore, osteoclasts break down bone. BCX interferes with synthesis of osteoclasts and vitamin K2 causes of apoptosis of those osteoclasts that are formed. Hence, these nutrients would tend to reduce bone resorption. In the presence of calcium, and optionally vitamin D, bone formation would then balance or exceed bone resorption to enhance bone density, bone strength, reduce fragile bones, and reducing biomarkers of bone resorption (tartrate-resistant acid phosphatase activity—TRAP- and the C-terminal telopeptides of type I collagen), all of which are key indicators of osteoporosis. The benefits of reducing the required amount of calcium required to increase bone strength, bone density, thus reducing osteoporosis, and affect biomarkers of bone formation positively include providing effective products with increased compliance. The combination of vitamin k, in any form, BCX, calcium and vitamin D act synergistically to enhance bone formation; prevent and/or decrease bone fracture; prevent and/or decrease bone loss; increase BMD; increase bone strength; increase the biomarkers of bone formation (i.e., increasing bone-specific alkaline phosphatase, osteocalcin, and reducing undercarboxylated osteocalcin); as well as prevent and/or decrease bone resorption.

In a preferred embodiment, the vitamin D is vitamin D₃. In one embodiment the amount of vitamin D₃ to be administered usually will be from about 200 IU to about 2000 IUs. In a more preferred embodiment, the amount of vitamin D₃ present in the composition in the range from about 400 IU/day to about 800 IU/day.

In yet another embodiment, the composition comprising vitamin K2, BCX, and vitamin D act synergistically such that the amount vitamin K2, BCX, and/or vitamin D is reduced up to about 90%.

In still yet another embodiment, the composition comprising vitamin K2, BCX, calcium and vitamin D act synergistically such that the amount of vitamin K2, BCX, and/or vitamin D is reduced up to about 90%.

Additional ingredients that can be optionally included in the compositions of the present invention include other multivitamins and/or nutrients whose effects are known to impact osteoporosis. For example, homocysteine levels increase with age and it has been shown that several chronic diseases also are associated with increased levels, including osteoporosis. High homocysteine levels are associated with osteoporosis by interfering with collagen cross-linking. Homocysteine can be reduced with nutrients vitamins B12, B6, folic acid, and riboflavin. Hence, the compositions of the invention can be combined with these nutrients as well. The composition of the present invention can also include various minerals known to positively affect the health of bone. Such minerals include magnesium, copper, zinc, phosphorus, manganese, boron, fluoride, silicon, and chromium. These minerals can be present as pharmaceutically acceptable salts or, in some cases, as bioavailable chelates. The term “pharmaceutically acceptable salt” refers to a salt prepared from pharmaceutically acceptable non-toxic acids or bases including inorganic or organic acids. Examples of such inorganic acids are hydrochloric, hydrobromic, hydroiodic, sulfuric, and phosphoric acids. Appropriate organic acids may be selected, for example, from aliphatic, aromatic, carboxylic and sulfonic classes of organic acids, examples of which are formic, acetic, propionic, succinic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic, stearic, sulfanilic, algenic, and galacturonic. In addition, some minerals can be provided in more bioavailable forms, such as amino acid chelates, which are well known in the art. U.S. Pat. No. 5,292,538. Examples of minerals that can be provided as amino acid chelates include calcium, magnesium, manganese, zinc, boron, copper, and chromium. Preferred magnesium salts and chelates include magnesium oxide, magnesium glycinate, magnesium citrate, magnesium aspartate, and magnesium malate, and mixtures thereof. Preferred copper salts and chelates include copper sulfate, copper oxide, copper glycinate, copper lysinate, copper tyrosinate, and copper gluconate, and mixtures thereof. Preferred zinc salts and chelates include zinc sulfate, zinc oxide, zinc ascorbate, zinc glycinate, zinc aspartate, zinc arginate, zinc citrate, zinc gluconate, and zinc picolinate, and mixtures thereof. Preferred manganese salts and chelates include manganese sulfate, manganese glycinate, manganese gluconate, manganese arginate, and manganese aspartate, and mixtures thereof. Preferred boron salts and chelates include boron citrate, boron aspartate, and boron glycinate, and mixtures thereof. Preferred fluoride salts include a neutral sodium fluoride and an acidulated phosphate fluoride, and mixtures thereof. Preferred silicon salts and chelates include silicon dioxide and Horse herb (Equisetum arvense), and mixtures thereof. Preferred chromium salts and chelates include dinicotinate glycinate, chromium aspartate, and chromium picolinate, and mixtures thereof. Such nutrients are well known in the art.

Nutritionally complete compositions contain all vitamins and minerals understood to be essential in the daily diet and these should be present in nutritionally significant amounts. Those skilled in the art appreciate that minimum requirements have been established for certain vitamins and minerals that are known to be necessary for normal physiological function.

The composition of the invention may optionally contain vitamins and mineral ingredients for nutritional supplementation. One skilled in the art would understand that appropriate additional amounts (overages) of vitamin and mineral ingredients need to be provided to compensate for some loss during processing and storage of such compositions. To select a specific vitamin or mineral compound to be used in the composition requires consideration of that vitamin or mineral compound's chemical nature is compatible with the formulation, processing and shelf storage.

Bone enhancing agents, known in the art to increase bone formation, bone density or bone mineralization, or to prevent bone resorption may be used in the methods and pharmaceutical compositions of the invention. Suitable bone enhancing agents include natural or synthetic hormones, such as estrogens, androgens, calcitonin, prostaglandins and parathormone; growth factors, such as platelet-derived growth factor, insulin-like growth factor, transforming growth factor, epidermal growth factor, connective tissue growth factor and fibroblast growth factor; vitamins, minerals, such as aluminum, strontium and fluoride; statin drugs, including pravastatin, fluvastatin, simvastatin, lovastatin and atorvastatin; agonists or antagonist of receptors on the surface of osteoblasts and osteoclasts, including parathormone receptors, estrogen receptors and prostaglandin receptors; bisphosphonate and anabolic bone agents.

In a more preferred embodiment, the composition of the current invention can be administered in combination with other bone effecting ingredients, including but not limited to estrogens, calcitonin, selective estrogen receptor modulators (SERMS), and/or bisphosphonates. These compositions can be used in the treatment and/or prevention bone loss, increasing bone density, increasing bone strength, increasing biomarkers of bone formation (i.e., increasing bone-specific alkaline phosphatase, osteocalcin, and reducing undercarboxylated osteocalcin) and reducing biomarkers of bone resorption (tartrate-resistant acid phosphatase activity—TRAP- and the C-terminal telopeptides of type I collagen). In one embodiment, the compositions further comprise estrogens and/or conjugated estrogens, including but not limited to Prempro® (conjugated estrogens+medroxyprogesterone) or Premarin® (conjugated estrogens). Available treatments of estrogens and conjugated estrogens include dosages of about 0.625 and about 0.25 mg per day and transdermal estrogen (a weekly patch containing 0.05 mg). In another embodiment, the compositions further comprise calcitonin, including but not limited to Calcimar® and/or Miacalcin®, a hormone that directly inhibits osteoclastic bone resorption. Calcitonin dosing is about 200 IU per day or about 50 to about 100 IU per day given intranasally or by intramuscular injection. In another embodiment, the compositions may contain bisphosphonates, including but not limited to alendronate (Fosamax®), risedronate (Actonel®), etidronate (Didronel®), and/or ibandronate (Boniva®), or any combination thereof. All bisphosphonates act similarly on bone in binding permanently to mineralized bone surfaces and inhibiting osteoclastic activity. Thus, less bone is degraded during the remodeling cycle. Dosing for alendronate is about 5 mg/day for prevention and about 10 mg/day for treatment by mouth. In yet another embodiment, the compositions of the invention include Selective Estrogen Receptor Modulators (SERMS), including but not limited to raloxifene (Evista®), tamoxifen (Nolvadex®), bazedoxifene (Viviant®), or any combination thereof. For example, raloxifene (60 mg/day by mouth) is the first drug from a new class—selective estrogen receptor modulators—to be studied in the treatment of osteoporosis. Raloxifene is thought to block estrogen in a similar manner while also binding and stimulating other estrogen tissue receptors. Raloxifene inhibits trabecular and vertebral bone loss by blocking the activity of cytokines, which stimulate bone resorption. Also contemplated within the scope of this invention are the optional inclusion of other bone-building drugs or supplements (phytoestrogens, or parathyroid hormones, including but not limited to teriparatide (Forteo®), administered alone or in combination with other osteoporosis treatments. One skilled in the art will appreciate that each of the classes of drugs listed above can be used either alone or in combination.

If desired, the compositions may be administered simultaneously or sequentially with other active ingredients. These active ingredients may, for example include other medicaments or compositions capable of improving bone health and/or prevention of bone disease. Such medicaments or compositions may, for example, be those of use in the treatment of osteoarthritis or osteoporosis.

The compositions of the invention are intended for use against target populations: osteoporotic or osteopenic due to: aging, endocrine imbalances (hyperthyroid, Paget's Disease), calcium and/or vitamin D deficient, loss of estrogen due to menopause, use of tobacco, take certain drugs that negatively impact bone strength (e.g., diurectics including furosemide [Lasix], bumetanide [Bumex], ethacrynic acid [Edecrin] and torsemide [Demadex]), which cause excretion of fluids and a loss of calcium; corticosteroid drugs, such as prednisone, cortisone, prednisolone and dexamethasone on a long-term basis; blood-thinning drug heparin, methotrexate, some antiseizure drugs and aluminum-contain antacids), or have had certain medical conditions or procedures that decrease calcium absorption including stomach surgery, Crohn's disease, anorexia nervosa or Cushing's disease (a rare disorder in which the adrenal glands produce too much corticosteroid hormones).

The inventors have surprisingly found that the compositions described herein are beneficial for the increased performance of calcium by improving calcium absorption into bone and facilitation of calcium binding into the bone matrix. The compositions can also aid in increasing the performance of vitamin D through the formation of osteoblasts, D is able to increase osteocalcin synthesis.

The invention is further directed to methods of treating and/or preventing the following: treat and/or prevent primary and secondary bone fractures; increase bone mineral density; strengthen the bone matrix (foundation, skeletal frame, quality); increase in bone strength; to decrease bone resorption (quantity & rate); to increase bone formation; improve bone healing; improve bone turnover; facilitate bone replacement/replenishment; reduce/counteract and/or prevent degenerative impact (of diseases or drugs) on bone; and to optimize bone peak mass; furthermore the compositions can be used to aid in the carboxylation of osteocalcin; the synthesis of osteoblasts. In osteoclasts, the compositions can be used to reduce induction, both initiation & differentiation and to promote apoptosis.

The compounds described herein may be used in the methods of the invention to enhance bone formation in a range of mammals, including humans. In an embodiment of the invention the mammal is a human in need of enhanced bone formation. In one aspect, the human in need has a bone deficit, which means that they will have less bone than desirable or that the bone will be less dense or strong than desired. A bone deficit may be localized, such as that caused by a bone fracture or systemic, such as that caused by osteoporosis. Bone deficits may result from a bone remodeling disorder whereby the balance between bone formation and bone resorption is shifted, resulting in a bone deficit. Examples of such bone remodeling disorders include osteoporosis, Paget's disease, osteoarthritis, rheumatoid arthritis, achondroplasia, osteochodrytis, hyperparathyroidism, osteogenesis imperfecta, congenital hypophosphatasia, fribromatous lesions, fibrous displasia, multiple myeloma, abnormal bone turnover, osteolytic bone disease and periodontal disease. Bone remodeling disorders includes metabolic bone diseases that are characterized by disturbances in the organic matrix, bone mineralization, bone remodeling, endocrine, nutritional and other factors which regulate skeletal and mineral homeostasis. Such disorders may be hereditary or acquired and generally are systemic affecting the entire skeletal system.

Thus, in one aspect the human may have a bone remodeling disorder. Bone remodeling as used herein refers to the process whereby old bone is being removed and new bone is being formed by a continuous turnover of bone matrix and mineral that involves bone resorption by osteoclasts and bone formation by osteoblasts. Osteoporosis is a common bone remodeling disorder characterized by a decrease in bone density of normally mineralized bone, resulting in thinning and increased porosity of bone cortices and trabeculae. The skeletal fragility caused by osteoporosis predisposes sufferers to bone pain and an increased incidence of fractures. Progressive bone loss in this condition may result in a loss of up to 50% of the initial skeletal mass. Primary osteoporosis includes idiopathic osteoporosis which occurs in children or young adults with normal gonadal function, Type I osteoporosis, also described as post-menopausal osteoporosis, and Type II osteoporosis, senile osteoporosis, occurs mainly in those persons older than 70 years of age. Causes of secondary osteoporosis may be endocrine (e.g. glucocorticoid excess, hyperparathyroidism, hypoganodism), drug induced (e.g. corticosteroid, heparin, tobaco) and miscellaneous (e.g. chronic renal failure, hepatic disease and malabsorbtion syndrome osteoporosis). The phrase “at risk of developing a bone deficit”; as used herein, is intended to embrace mammals and humans having a higher than average predisposition towards developing a bone deficit. As an example, those susceptible towards osteoporosis include post-menopausal women, elderly males (e.g. those over the age of 65) and those being treated with drugs known to cause osteoporosis as a side-effect (e.g. steroid-induced osteoporosis). Certain factors are well known in the art which may be used to identify those at risk of developing a bone deficit due to bone remodeling disorders like osteoporosis. Important factors include low bone mass, family history, life style, estrogen or androgen deficiency and negative calcium balance. Postmenopausal women are particularly at risk of developing osteoporosis. Hereinafter, references to treatment of bone diseases are intended to include management and/or prophylaxis except where the context demands otherwise.

The methods of the invention may also be used to enhance bone formation in conditions where a bone deficit is caused by factors other than bone remodeling disorders. Such bone deficits include fractures, bone trauma, conditions associated with post-traumatic bone surgery, post-prosthetic joint surgery, post plastic bone surgery, post dental surgery, bone chemotherapy, post dental surgery and bone radiotherapy. Fractures include all types of microscopic and macroscopic fractures. Examples of fractures includes avulsion fracture, comminuted fracture, transverse fracture, oblique fracture, spiral fracture, segmental fracture, displaced fracture, impacted fracture, greenstick fracture, torus fracture, fatigue fracture, intraarticular fracture (epiphyseal fracture), closed fracture (simple fracture), open fracture (compound fracture) and occult fracture.

As previously mentioned, a wide variety of bone diseases may be treated in accordance with the present invention, for example all those bone diseases connected with the bone-remodelling cycle. Examples of such diseases include all forms of osteoporosis, osteomalacia, rickets and Paget's disease. Osteoporosis, especially of the post-menopausal, male and steroid-induced types, is of particular note. In addition, the above described compounds find use as antiresorption agents generally, as bone promotion agents and as anabolic bone agents. Such uses form another aspect of the present invention.

The compositions of the invention may be administered in the form of a pharmaceutical composition comprising vitamin K², and BCX, and optionally calcium and/or vitamin D, in admixture or association with a pharmaceutically acceptable carrier or diluent. The compositions may be formulated into a composition suitable for administration by any convenient route, e.g. orally (including sublingually), parenterally (including intravenous, intramuscular, intraperitoneal and subcutaneous administration) and rectally, oral administration being preferred. Orally administrable compositions may, if desired, contain one or more physiologically compatible carriers and/or excipients and may be solid or liquid. The compositions may take any convenient form including, for example, tablets, coated tablets, capsules, lozenges, aqueous or oily suspensions, solutions, emulsions, syrups, elixirs and dry products suitable for reconstitution with water or another suitable liquid vehicle before use. The compositions may advantageously be prepared in dosage unit form. Tablets and capsules according to the invention may, if desired, contain conventional ingredients such as binding agents, for example syrup, acacia, gelatin, sorbitol, tragacanth or polyvinyl-pyrollidone; fillers, for example lactose, sugar, maize-starch, calcium phosphate, sorbitol or glycine; lubricants, for example magnesium stearate, talc, polyethylene glycol or silica; disintegrants, for example potato starch; or acceptable wetting agents such as sodium lauryl sulphate. Tablets may be coated according to methods well known in the art.

Liquid compositions may contain conventional additives such as suspending agents, for example sorbitol syrup, methyl cellulose, glucose/sugar syrup, gelatin, hydroxymethylcellulose, carboxymethylcellulose, aluminum stearate gel or hydrogenated edible fats; emulsifying agents, for example lecithin, sorbitan monooleate or acacia; non-aqueous vehicles, which may include edible oils, for example vegetable oils such as arachis oil, almond oil, fractionated coconut oil, fish-liver oils, oily esters such as polysorbate 80, propylene glycol, or ethyl alcohol; and preservatives, for example methyl or propyl p-hydroxybenzoates or sorbic acid. Liquid compositions may conveniently be encapsulated in, for example, gelatin to give a product in dosage unit form.

Formulations for oral delivery may be formulated in a delayed release formulation. Delayed release formulations are well known in the art and include for example, delayed release capsules or time pills, osmotic delivery capsules etc.

Compositions for parenteral administration may be formulated using an injectable liquid carrier such as sterile pyrogen-free water, sterile peroxide-free ethyl oleate, dehydrated alcohol or propylene glycol or a dehydrated alcohol/propylene glycol mixture, and may be injected intravenously, intraperitoneally, subcutaneously or intramuscularly.

Compositions for rectal administration may be formulated using a conventional suppository base such as cocoa butter or another glyceride.

It is also contemplated within the scope of the invention, the compositions can be delivered as a fortified food and/or a beverage product. Examples of such food and/or beverage products include, but are not limited to, juice drinks, dairy drinks, powdered drinks, sports drinks, mineral water, soy beverages, hot beverages, nutritional supplement drinks (Ensure®, etc.) diet drinks, diet bars, prepared meals, candy, snack products, prepared meat products, milk, cheese, yogurt, bread, cereal and any other suitable food and/or beverage. One skilled in the art would appreciate the preferred dosage form for said food and/or beverage product.

The composition of the invention can be packaged in any type of container known in the art to be useful for storing nutritional products such as glass, lined paperboard, plastic, coated metal cans and the like, but are most preferably packaged in a bottle for tube feeding, and in a paper container for oral use.

It will be understood that the dosages of compositions and the duration of administration according to the invention will vary depending on the requirements of the particular subject. The precise dosage regime will be determined by the attending physician who will, inter alia, consider factors such as body weight, age and symptoms (if any). The compositions may if desired incorporate one or more further active ingredients. During the dosing regimen, administration may be effected once or more times per day, for example once, twice, three or four times per day.

The following examples are provided to better define and specifically delineate the compositions of the present invention so as to better enable one skilled in the art how to prepare and formulate them. They are for illustrative purposes only however, and it is recognized that minor changes and variations may be made with respect to the specific ingredients, their amounts and the manner of formulating them. It therefore should be understood that to the extent any such variations do not materially alter or change the characteristics of said formulations, such changes are deemed as falling within the spirit and scope of the invention as defined by the claims that follow.

### EXAMPLES

One embodiment of the invention comprises:

| | | |
|---|---|---|
| Vitamin K2, MK7 | 50 | μg |
| BCX | 2.5 | mg |

1. A composition comprising vitamin K2 and betacryptoxanthin.

2. The composition of claim 1, wherein the vitamin K2 is MK4 or MK7.

3. The composition of claim 2, wherein the vitamin K2 is MK7.

4. A composition comprising vitamin K2, calcium, betacryptoxanthin and vitamin D.

5. The composition of claim 4, wherein the vitamin K2 is MK4 or MK7.

6. The composition of claim 4, wherein the vitamin K2 is MK7.

7. The composition of claim 5, wherein the vitamin D is selected from the group consisting of vitamin D₃, vitamin D₂, and their biologically active metabolites and precursors.

8. A method for enhancing and/or improving bone formation in a human subject in need thereof comprising administering to said mammal an effective amount of a composition comprising vitamin K2 and betacryptoxanthin.

9. The method of claim 8, wherein the vitamin K2 is MK4 or MK7.

10. The method of claim 9, wherein the vitamin K2 is MK7.

11. The method of claim 8, wherein the composition further comprises an effective amount of vitamin D.

12. The method of claim 11, wherein the vitamin D is selected from the group consisting of vitamin D₃, vitamin D₂, and their biologically active metabolites and precursors.

13. The method of claim 8, wherein the composition further comprises an effective amount of valcium.

14. A method for preventing and/or reducing bone mineral density loss in a human subject in need thereof comprising administering to said mammal an effective amount of a composition comprising an effective amount of a composition comprising vitamin K2 and betacryptoxanthin.

15. The method of claim 14, wherein the vitamin K2 is MK4 or MK7.

16. The method of claim 15, wherein the vitamin K2 is MK7.

17. The method of claim 14, wherein the composition further comprises an effective amount of vitamin D.

18. The method of claim 17, wherein the vitamin D is selected from the group consisting of vitamin D₃, vitamin D₂, and their biologically active metabolites and precursors.

19. The method of claim 14, wherein the composition further comprises an effective amount of calcium.

20. A method for the prevention and/or treatment of osteoporosis in a human subject in need thereof comprising administering to said mammal an effective amount of a composition comprising vitamin K2 and betacryptoxanthin.

21. The method of claim 20, wherein the vitamin K2 is MK4 or MK7.

22. The method of claim 21, wherein the vitamin K2 is MK7.

23. The method of claim 20, wherein the composition further comprises an effective amount of vitamin D.

24. The method of claim 23, wherein the vitamin D is selected from the group consisting of vitamin D₃, vitamin D₂, and their biologically active metabolites and precursors.

25. The method of claim 20, wherein the composition further comprises an effective amount of calcium.

26. A method for increasing and/or improving bone strength in a human subject in need thereof comprising administering to said mammal an effective amount of a composition comprising vitamin K2 and betacryptoxanthin.

27. The method of claim 26, wherein the vitamin K2 is MK4 or MK7.

28. The method of claim 27, wherein the vitamin K2 is MK7.

29. The method of claim 26, wherein the composition further comprises an effective amount of vitamin D.

30. The method of claim 29, wherein the vitamin D is selected from the group consisting of vitamin D₃, vitamin D₂, and their biologically active metabolites and precursors.

31. The method of claim 29, wherein the composition further comprises calcium.

32. A composition comprises vitamin K2, betacryptoxanthin and calcium.

33. The composition of claim 32, wherein the vitamin k2 is MK4 or MK7.

34. The composition of claim 33, wherein the vitamin k2 is MK7.
